(19) United States
(12) Patent Application Publication
(10) Pub. No.: US 2004/0260129 A1
(43) Pub. Date: Dec. 23, 2004

(54) 6-(2,7-octadienyl)-1,4-cyclooctadiene

(76) Inventors: Norbert Wilczok, Muelheim (DE); Matthias May, Dorsten (DE); Martin Knaack, Duelmen (DE)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/831,159

(22) Filed: Apr. 26, 2004

(30) Foreign Application Priority Data
Apr. 25, 2003 (DE) 103 18 773.1

(51) Int. Cl. C07C 13/00; C07C 13/02

(52) U.S. Cl. 585/20; 585/370; 525/242

(57) ABSTRACT

A polyunsaturated hydrocarbon, 6-(2,7-octadienyl)-1,4-cyclooctadiene. 6-(2,7-Octadienyl)- 1,4-cyclooctadiene is prepared by reacting butadiene-1,3 in the presence of a catalyst system consisting of a nickel complex, an organic phosphite and dialkylaluminum alkoxide, removing unreacted butadiene-1,3 starting material and cyclooctadiene and cyclododecatriene material formed in the reaction from the reaction product obtained, and fractionally distilling 6-(2,7-octadienyl)-1,4-cyclooctadiene from the material obtained after the distillative separation of butadiene-1,3, cyclooctatriene and C₁₂ compounds.

### BACKGROUND OF THE INVENTION

1. Field of the Invention

The invention relates to the polyunsaturated C₁₆ hydrocarbon 6-(2,7-octadienyl)-1,4-cyclooctadiene, to its preparation from butadiene-1,3 and to its use. Potential fields of use for this high-boiling C₁₆ hydrocarbon are in the perfume and aroma industry, for example in the conversion to macrocyclic ketones (Takasago Perfumery Co., JP 57 021 254, cited according to CAN 97: 162 457) or else as a starting product for the preparation of pharmaceutical products. However, one of the most important area of application is as a crosslinker in synthetic rubbers in rubber production, for example for tires, or as a copolymer in the production of plastics, for example, polyolefins.

2. Description of the Background

Starting from butadiene-1,3, monomeric hydrocarbons having an even number of carbon atoms can be prepared. Typical monomers are compounds having from 12 to 24 carbon atoms, consisting, in particular, of a ring structure having from 6 to 16 carbon atoms and optionally one or more side chains.

Important, nonlimiting representatives of such monomers include 3-(2-butenyl)-1,5,9-cyclododecatriene [34057-87-9], 3-(3-butenyl)-1,5,9-cyclododecatriene [34057-86-8] and 3-(1-methylpropenyl)-1,5,9-cyclododecatriene [34057-85-7] and cyclohexadeca-1,5,9,13-tetraene [23579-21-7].

While there are several known processes for synthesizing cyclohexadeca-1,5,9,13-tetraene starting from butadiene over nickel catalysts (GB 1 287 252, Toray Industries; DE 19 06 361, Toyo Rayon Co.) or by ring-opening metathesis from 1,5-cyclododecadiene over tungsten catalysts (E. A. Ofstead, Macromol. Synth. 1977, 6, 69; U.S. Pat. No. 3,439,057, Goodyear Tire & Rubber Co.), rhodium catalysts (K. Sato et al., Bull. Soc. Chem. Jpn. 1979, 52, 3192) or rhenium catalysts (U.S. Pat. No. 3,865,888, Goodyear Tire), the other monomer components have hitherto hardly been described in the literature. Only Mitsubishi Petrochemicals Co. Ltd. has described the preparation of such monomers over Ziegler catalysts starting from a mixture of 11,3-butadiene and dimers, for example 1,3,7-n-octatriene. Patent documents on this subject are DE 20 63 348, U.S. Pat. No. 3,658,926 and JP 48 019 304 (cited according to CA: 79:78229). Apart from cyclododecatriene, the compounds described are in particular C₁₋₆ hydrocarbons which have a C₁₂ ring structure.

### SUMMARY OF THE INVENTION

Accordingly, one object of the present invention is to prepare cyclic hydrocarbons, in particular, those having relatively short side chains, and having property profiles which modify and supplement those already known in an advantageous manner.

Briefly, this object and other objects of the present invention as hereinafter will become more readily apparent can be attained by a process for preparing 6-(2,7-octadienyl)-1,4-cyclooctadiene from butadiene-1,3, which comprises converting butadiene-1,3 over a catalyst system consisting of a nickel complex, an organic phosphite and dialkylaluminum alkoxide to a reaction product containing 6-(2,7-octadienyl)-1,4-cyclooctadiene, distilling residual butadiene-1,3, and cyclooctadiene and cyclododecatriene which have formed from the reaction product, and fractionally distilling 6-(2,7-octadienyl)-1,4-cyclooctadiene from the remaining material under reduced pressure.

### DETAILED DESCRIPTION OF THE PREFERRED EMBODIMENTS

It has now been found that, surprisingly, in the synthesis of cyclooctadiene from butadiene-1,3,6-(2,7-octadienyl)-1,4-cyclooctadiene, a C₁₆ hydrocarbon having a C₈ ring, which has not been known in the past, can be prepared. This compound is suitable as a starting component, for example, in the preparation of fragrances, as a crosslinker, for example in the rubber field, or as copolymer, for example in polyolefins. The synthesis succeeds in particular with a catalyst system which consists of a nickel complex, an organic phosphite and an activating dialkylaluminum alkoxide.

Suitable nickel compounds are compounds which result in nickel(0) compounds by activation (reduction). These include primarily nickel acetylacetonate.

The phosphites preferably contain aromatic groups. Particularly suitable are tris-(o-phenylphenyl) phosphite and/or tris(2,4-di-tert-butylphenyl) phosphite.

The alkyl groups of the activating dialkylaluminum component are radicals having from 1 to 4, preferably 2, carbon atoms. Suitable examples include the methyl group, but in particular the ethyl, n- and isopropyl, and butyl groups in their various isomeric forms. Mixed dialkyl compounds can also be used. The alkoxide radical likewise contains from 1 to 4, preferably 2, carbon atoms, and the aforementioned list applies in the same sense. A particularly preferred dialkylaluminum alkoxide is diethylaluminum ethoxide.

After the conversion of the butadiene-1,3 and the removal by distillation of any unconverted butadiene-1,3 and cyclooctadiene in a vacuum stage (flash evaporation), a product mixture remains in the bottom and contains C₁₂, C₁₆ and C₂₀ hydrocarbons in addition to other high-boilers, from which C₁₂ compounds can be separated from C₁₆ compounds in a vacuum distillation step. The product 6-(2,7-octadienyl)-1,4-cyclooctadiene (b.p. 127° C. at 3 mbar) can be isolated and is surprisingly very stable.

Having now generally described this invention, a further understanding can be obtained by reference to certain specific examples which are provided herein for purposes of illustration only and are not intended to be limiting unless otherwise specified.

Preparation of the Pure Component

Butadiene-1,3 was initially reacted in the presence of nickel acetylacetonate, tris(o-phenylphenyl) phosphite and diethylaluminum ethoxide. After removal by distillation of unconverted butadiene, and of cyclooctadiene and cyclododecatriene which had formed, 6-(2,7-octadienyl)-1,4-cyclooctadiene was isolated under reduced pressure at 2-6 mbar and 125-135° C.

Analysis for Purity

Analysis of the substance for purity was conducted with a gas chromatograph having a flame ionization detector; Supelcowax TM 10, length 30 meters.

The product is liquid, water-clear and has virtually no odor at 20° C. (room temperature).

Substance Characterization

One- and two-dimensional homo- and heteronuclear NMR spectra (¹H and ¹³C NMR spectra) and also infrared spectra were obtained to identify and elucidate the structure of the C₁₆H₂₄ hydrocarbon 6-(2,7-octadienyl)-1,4-cyclooctadiene of the following structure:

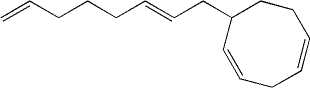

The NMR Spectroscopy Characterization of the Structure was by H,H-COSY, HMQC, HMBC, 1,1-ADEQUATE and in Particular by INADEQUATE Experiments. The Cis-Arrangement of the Two Double Bonds in the Ring was Supported by a ¹H NMR Experiment with Selective Decoupling. The Existence of a Trans-Double Bond was Proved by IR Spectroscopy.

The disclosure of German priority application Serial No. 103 18 773.1 filed Apr. 25, 2003, is hereby incorporated by reference into the present application.

Obviously, numerous modifications and variations of the present invention are possible in light of the above teachings. It is therefore to be understood that within the scope of the appended claims, the invention may be practiced otherwise than as specifically described herein.

What is claimed is:

1. 6-(2,7-Octadienyl)-1,4-cyclooctadiene.

2. A process for preparing 6-(2,7-octadienyl)-1,4-cyclooctadiene from butadiene-1,3, which comprises:
converting butadiene-1,3 in the presence of a catalyst system consisting of a nickel complex, an organic phosphite and dialkylaluminum alkoxide;
distilling residual butadiene-1,3, and cyclooctadiene and cyclododecatriene, which have formed in the reaction, from the reaction product obtained; and
fractionally distilling 6-(2,7-octadienyl)-1,4-cyclooctadiene from the residue obtained after the distillative separation of butadiene-1,3, cyclooctatriene and C₁₂ compounds.

3. The process of claim 2, wherein said nickel complex is nickel acetylacetonate.

4. The process of claim 2, wherein the phosphite component of the catalyst is tris(o-phenylphenyl) phosphite, tris(2,4-di-tert-butylphenyl) phosphite or a combination thereof.

5. The process of claim 2, wherein the dialkylaluminum alkoxide of the catalyst is a C₁₋₄-dialkylaluminum alkoxide.

6. The process of claim 2, wherein the dialkylaluminum alkoxide of the catalyst is a C₁₋₄-dialkylaluminum ethoxide.

7. The process of claim 6, wherein the dialkylaluminum alkoxide of the catalyst is diethylaluminum ethoxide.

8. The process of claim 2, wherein 6-(2,7-octadienyl)-1,4-cyclooctadiene is obtained by vacuum distillation.

9. A method of preparing a fragrance material, comprising:
conducting the preparation of a fragrance employing 6-(2,7-octadienyl)-1,4 cyclooctadiene as a starting material in the preparation.

10. A method of preparing a pharmaceutical product, comprising:
conducting the preparation of a pharmaceutical product employing 6-(2,7-octadienyl)-1,4-cyclooctadiene as a starting material in the preparation.

11. A method of preparing a rubber product, comprising:
crosslinking a rubber material with 6-(2,7-octadienyl)-1,4-cyclooctadiene as a crosslinking agent.

12. A method of producing a plastic, comprising:
preparing a plastic by conducting a polymerization reaction with 6-(2,7-octadienyl)-1,4-cyclooctadiene as a comonomer.
